(19) United States
(12) Patent Application Publication
(10) Pub. No.: US 2002/0165427 A1
(43) Pub. Date: Nov. 7, 2002

(54) Intravesicular device

(76) Inventors: Daniel Yachia, Herzliya (IL); Eran Hirszowicz, Ramat-Chen (IL)

Correspondence Address: LADAS & PARRY, 26 WEST 61ST STREET, NEW YORK, NY, 10023, US

(21) Appl. No.: 09/525,294

(22) Filed: Mar. 14, 2000

Related U.S. Application Data
(63) Continuation-in-part of application No. 09/268,109, filed on Mar. 15, 1999, now Pat. No. 6,293,923.
(63) Continuation-in-part of application No. 09/363,287, filed on Jul. 28, 1999.

(51) Int. Cl. A61F 2/02

(52) U.S. Cl. 600/31; 604/517; 604/96.01

(57) ABSTRACT

A system for treating a urinary bladder of an individual. The system comprises a resiliently flexible, solid body for insertion into the urinary bladder. The system also comprises an applicator for inserting the body into the urinary bladder or for removing the body from the urinary bladder. The system is useful in treating the urinary bladder, in monitoring the urinary bladder and in the treatment of urinary incontinence.

### FIELD OF THE INVENTION

The invention is in the field of medical devices. More specifically, the invention relates to devices for the treatment of urinary incontinence.

### BACKGROUND OF THE INVENTION

Several disorders of the urinary tract are known. Among these are urinary incontinence, chronic urinary tract infections, urinary bladder tumors.

Urinary Incontinence

Urinary incontinence mostly affects women (approximately 10 million in the U.S.A. alone) primarily after childbirth or due to old age. In men, urinary incontinence often occurs as a complication of surgery or old age (approximately 3 million in the U.S.A.).

Incontinence has serious economic, health, social and psychological consequences. Its estimated cost to the health system in the United States in 1993 was US $16 billion. It leads to chronic and severe skin irritation in the genital area, an increase in urinary infections and urosepsis. Fear of incontinence and odors in public cause incontinent people to severely restrict their social activities. The impact on the mental health of the affected people may be even more devastating than the social and health consequences. They suffer severe embarrassment, loss of self-esteem, depression and anxiety.

Urinary incontinence can be divided into four groups:

Stress Incontinence—is the involuntary release of urine due to a sudden increase in the intra-abdominal pressure caused by laughing, sneezing, coughing, running, etc. This is the most common type of incontinence and in women may be the result of anatomical changes in the pelvic organs after childbirth, estrogen deficiency, unsuccessful surgical repairs for incontinence or pelvic irradiation. In men, it often happens after surgery for benign enlargement of the prostate gland or after radical removal of the prostate.

Total Incontinence—is the continuous leak of urine entering the bladder due 15 to failure of the sphincteric muscles.

Urge Incontinence—is involuntary loss of urine due to involuntary bladder contractions. This type of incontinence mostly affects the elderly who leak until they reach a toilet.

Mixed Incontinence—is a combination of stress and urge incontinence. This condition is more common in elderly women than men.

Ideally, treatment of incontinence should provide permanent dryness and is easy to perform.

Pharmacological treatments of bladder dysfunctions are based either on estrogen replacement for treating post-menopausal vaginal and urethral atrophy or on agents affecting the tonus of the bladder muscle. Since affected elderly women suffer from both hormonal deficiency and urinary incontinence, both types of agents are usually prescribed simultaneously.

Surgical treatments are based on restoring the anatomical changes causing the incontinence. Although in the short-term most surgical procedures restore continence, the long-term prognosis is usually unsatisfactory. Moreover, surgery entails morbidity and high expenses.

Conservative/behavioral treatments are based on pelvic floor muscle exercises, bladder training, biofeedback, vaginal cones, low-frequency electrostimulation of pelvic floor muscles, intravaginal bladder neck support pessaries, urethral meatus suction cups and intraurethral devices. Conservative treatments are time consuming and require the patients' understanding, cooperation and persistence.

Devices which have been used to obtain almost immediate dryness in incontinent people can be divided into two groups:

(1) Urethral Plugs/Inserts

These comprise a flexible rod having, a 14 Ch. (approximately 4.5 mm) diameter and a length adjusted to fit the length of the patient's urethra. The rod has an inflatable device on its bladder end and a flange at other end. After insertion of the device, the device is inflated in the bladder. The device and the flange, maintain the device in its proper position within the urethra. The device and rod form a mechanical barrier to retain the urine within the bladder. The device must be deflated and the device removed and discarded prior to voiding. Such inserts are known in the art, for example, the device known as RELIANCE produced by UroMed Corp., U.S.A. Since inserts are discarded after each voiding and replaced with a new one by the patient, manual dexterity of the patient is required. Insertion of an insert into a female has the risk of pushing vaginal and perineal bacteria into the bladder and insertion of an insert a few times a day increases this risk. The inconvenience of removing and inserting a new device and its costs, in addition to the infection risk, are the major disadvantages of these devices.

(2) Valve Catheters

These comprise a tube with a valve at one end. The bladder end of the device typically has a device or flanges for retaining the device in place and a flange at the other end to prevent migration into the bladder. The valve is opened for voiding through the lumen of the catheter with the help of an external magnet. The tube typically has a 18 Ch. (6 mm.) to 20 Ch. (approximately 7 mm) diameter and a length adjusted to fit the patient's urethra. For male incontinence, an active intraurethral Foley-type catheter is used. This device has a retaining, device at its bladder end and another smaller device under the prostate for fixing the device in place. The magnet activated valve is situated at the end of the device near the distal end of the urethra. Active inserts are typically left indwelling up to 4 weeks and are then replaced.

Examples of such catheters are disclosed in U.S. Pat. Nos. 5,030,199 and 5,234,409. Valve catheters are more convenient for the patient than the inserts; however, in females they cause ascending infection because they connect the bladder with the vulva which is rich in pathogenic bacteria, especially Escherichia Coli. Even with continuous use of antibiotics, infection is inevitable in the majority of cases. During prolonged use of cathetersor inserts in female patients, a relaxation of the urethra occurs and the patients may start to leak around the device. Unfortunately valve catheters and inserts are unavailable in increasing diameters.

A significant disadvantage of both the inserts and the valve catheters is the discomfort felt by the patient especially when sitting and during sexual intercourse (felt by the patient and the partner).

The present invention therefore provides a device for the treatment of urinary incontinence in which the disadvantages of the prior art devices are substantially reduced or eliminated.

(3) Urinary Bladder Plugs

U.S. Pat. No. 4,850,963 to Sparks et at. discloses a bolus for insertion in to a urinary bladder for the treatment of urinary incontinence. The bolus contains a ferromagnetic material and has a specific gravity greater an that of urine. The bolus is maintained at the urinary bladder outlet to the urethra under the influence of gravity so as to prevent the flow of urine into the urethra. For voiding, the bolus is displaced from the opening using an external magnet.

Urinary Tract Infections

Nearly half of all women experience urinary tract infection (UTI) at some point in their lifetime and most of these infections are confined to the bladder. Isolated UTIs can be treated by short and effective antibiotic treatment. However, recurrent UTIs often occur in women due to antibiotic resistant bacteria. In this case complicated infections often exhibit multidrug resistance and necessitate longer antimicrobial drug administrations.

Treatment of UTI often requires urinary levels of antimicrobial drugs that are several hundred times greater than those allowable in the blood. Many antibacterials cannot be used in UTI because, when taken orally or intravenously, they do not attain the required concentration in the urine, without exceeding the allowable limit in the blood. It would therefore be desirable to be able to continuously introduce antimicrobial drugs continuously and directly into the bladder,

Bladder tumors

Even after resection, bladder tumors may not only recur but may also invade deeper in the bladder wall. Due to the heterogenity of these tumors (from low-grade tumors showing a benign course to highly malignant high-grade tumors), there does not exist a single approach to the surveillance and treatment of these tumors. Intravesical drug therapies are often used for reducing tumor recurrence. In this approach, an immunotherapeutic or chemotherapeutic agent is inserted into the bladder through a catheter. This treatment is typically repeated once a week for 6 weeks and then once a month for a period of 6-12 months. However, periodic treatment has not been established as being effective in altering the progression of the tumor. Continuous local treatment with chemotherapeutic or radioactive materials may treat or prevent not only superficial tumors but also deep tumors as well. It would therefore be desirable to be able to introduce antitumoral drugs continuously and directly into the bladder.

Bladder dysfunction

During filling, the bladder muscle relaxes for keeping the intravesical pressure low while it contracts for voiding. Certain diseases such as spinal cord injuries. diabetes, multiple sclerosis, or hormonal changes after menopause or old age in both sexes may cause a hypo contractility or, paradoxically, hyper contractility of the muscle. In atonic bladder, pharmacological treatment is not very effective. In hyperreflexic bladder, drugs for relaxing the bladder cause constipation and mouth dryness and are therefore not tolerated well by the patients.

Diagnosis of bladder dysfunction requires continuously monitoring various bladder parameters during filling and/or voiding. These measurements usually are made by inserting a catheter connected to a measuring device into the bladder. This is done, for example, in uroflowmetry (measurement of urinary flow rate) which is non-invasive, simple and inexpensive. However, its sensitivity and specificity are low. Cystometry is an invasive technique for measuring bladder capacity, compliance and muscle tonus. Pressure-flow study is an invasive and costly test for distinguishing patients with low urinary flow due to obstruction or bladder antonia, from those with high intravesical pressure and high urinary flow. It is therefore a need in the art for a simple and inexpensive technique for intravesicular monitoring.

In the diagnostic procedure known as “urodynamics”, the bladder is filled through a catheter, and the response of the bladder is monitored. Available 24 hour urodynamic monitors have catheters or wires passing through the urethra, connecting sensors inserted into the bladder to a recorder. The connecting wires and catheters inadvertently introduce pathogenic bacteria from the genital areas into the bladder. It is therefore desirable to be able to monitor bladder function over several cycles of filling and voiding without the need for such wires or catheters.

Diagnosis of some intravesical pathological conditions often involves inserting an endoscope into the bladder and optically scanning the bladder walls. In cases of bleeding in the ureters or the kidneys, the observation of blood coming through the ureteral orifices allows determination of the origin of the bleeding. However, if the bleeding has temporarily stopped at the time of the examination, or if the blood concentration in the urine is insufficient to make the urine red or pink, endoscopy is of little value in reaching a diagnosis. In such cases more ivasive procedures are performed in order to enter the upper urinary tract. It is therefore desirable to be able to monitor the bladder over long periods of time.

Bladder shape during filling and its contraction during voiding is important for the diagnosis of certain bladder pathologies. These functions can be followed in fluoroscopy and by sonography. These techniques however are not accurate and cannot be used for monitoring changes in bladder shape over long periods of time. It would therefore be desirable to be able to continuously image the bladder interior over long periods of time.

The present invention therefore provides a device for continuous monitoring of the bladder interior and for the treatment of bladder disorders in which the disadvantages of the prior art devices are substantially reduced or eliminated.

### SUMMARY OF THE INVENTION

The present invention provides system comprising a solid body formed from a flexibly resilient material for insertion into the urinary bladder, and an applicator for inserting the body into the bladder. The body is compressed prior to insertion and then allowed to expand after insertion in the bladder.

The invention may be used for the intermittent sealing of the urinary bladder outlet and the prevention of involuntary urine leakage. Sealing the urinary bladder outlet involves positioning the device in the urinary bladder outlet so as to seal it. Unsealing the outlet to allow voiding of the bladder involves positioning the device away from the outlet. The body is preferably coated with a hydrophilic coating to reduce frictional forces between the body and the wall of the bladder. This facilitates release of the body from the outlet.

The invention may also be used for such purposes as for example, delivery of drugs, imaging the urinary bladder, and measuring intravesicular parameters such as pressure in the urinary bladder. When used for such purposes, the body may be, for example, positioned in the urinary bladder outlet or immobilized in some other desired location in the bladder.

The invention is entirely confined to the urinary bladder and has no urethral parts. As will become apparent in the description below, the body is easily inserted and removed. It may be left in the bladder for prolonged periods of time without encrusting or causing infections and is displaced within the bladder at will using a hand held magnet. The invention is comfortable for the patient and does not interfere with the daily activities of the patient including sitting, jogging, riding, or sexual intercourse.

Thus, in its first aspect the invention provides a urological medicinal system for use in medical procedures within a urinary bladder of an individual comprising:

(a) a resiliently flexible, solid body for insertion into the urinary bladder; and

(b) an applicator for inserting the body into the urinary bladder or for removing the body from the urinary bladder, the applicator fitted at an end thereof with a gripping device for releasably gripping the body.

In its second aspect, the invention provides a method for treating urinary incontinence in an individual comprising the steps of:

(a) compressing a resiliently flexible, solid body, formed with a magnetizable portion;

(b) inserting the body into a bladder of the individual;

(c) decompressing the body in the urinary bladder;

(d) displacing the body into a sealing position for sealing the urinary bladder; and

(e) displacing the body within the urinary bladder into an unsealing position for voiding the urinary bladder.

In its third aspect, the invention provides a method for releasing one or more substances into a urinary bladder comprising the steps of:

(a) loading the one or more substances into a solid, flexibly resilient body;

(b) compressing the body;

(c) inserting the body into the individual's urinary bladder; and

(d) decompressing the body in the urinary bladder.

In its fourth aspect, the invention provides a method for monitoring the interior of a urinary bladder comprising the steps of:

(a) compressing a solid flexibly resilient body comprising one or more devices for monitoring the urinary bladder;

(b) inserting the body into the individual's urinary-bladder;

(c) decompressing the body in the urinary bladder; and

(d) transmitting signals from at least one of the monitoring devices to a receiver.

In its fifth aspect, the invention provides a method for imaging the interior of a urinary bladder comprising the steps of:

(a) compressing a flexibly resilient body comprising a device for imaging the unary bladder,

(b) inserting the body into the urinary bladder;

(c) decompressing the body in the urinary bladder; and

(d) transmitting signals from the imaging device to a receiver.

In its sixth aspect, the invention provides a method for releasing one or more substances into a urinary bladder comprising steps of

(a) providing a body comprising a pump fed by a reservoir,

(b) loading the reservoir with the one or more substances;

(c) inserting the body into the urinary bladder; and

(d) activating the pump so as to release the one or more substances into the bladder.

In its seventh aspect, the invention provides a resiliently flexible, solid body for insertion into a urinary bladder.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows an embodiment of the body for use in the system of the invention having the appearance of an umbrella;

FIG. 2 shows longitudinal sections of umbrellas having a chamber for storing substances;

FIG. 3 shows an applicator for inserting an umbrella according to the invention into the urinary bladder of an individual;

FIG. 4 shows an expanded umbrella being inserted into the urinary bladder with an applicator;

FIG. 5 shows a retrieval device for retrieving the umbrella;

FIG. 6 shows use of a displacing member to displace the umbrella into a sealing position within the urinary bladder;

FIG. 7 shows use of a displacing member to displace the umbrella from a sealing position in the urinary bladder; and

FIG. 8 shows use of an immobilizing member.

FIG. 9 shows three other embodiments of the body for use in the system of the invention.

FIG. 10 shows an umbrella comprising an imaging device;

FIG. 11 shows an umbrella comprising devices for measuring bladder parameters;

### DETAILED DESCRIPTION OF SPECIFIC EMBODIMENTS

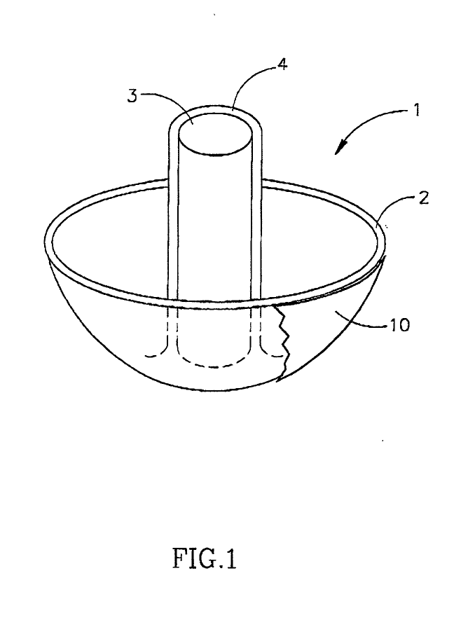

Reference is first made to FIG. 1 which shows an embodiment of the body for use in the system of the invention. The body, generally designated as 1, has in this embodiment a generally hemispherically shaped wall 2 and a stem 4 extending from the inner surface of wall 2. The body 1 is made of a resiliently flexible elastic biocompatible material. The body may be coated on its outer surface with a hydrophilic coating 10. The body 1 may optionally comprise a magnetizable portion. In the embodiment of the body 1 shown in FIG. 1, referred to herein as an “umbrella” the magnetizable may consist for example, of one or more metal particles associated with stem 4 or wall 2 of the umbrella.

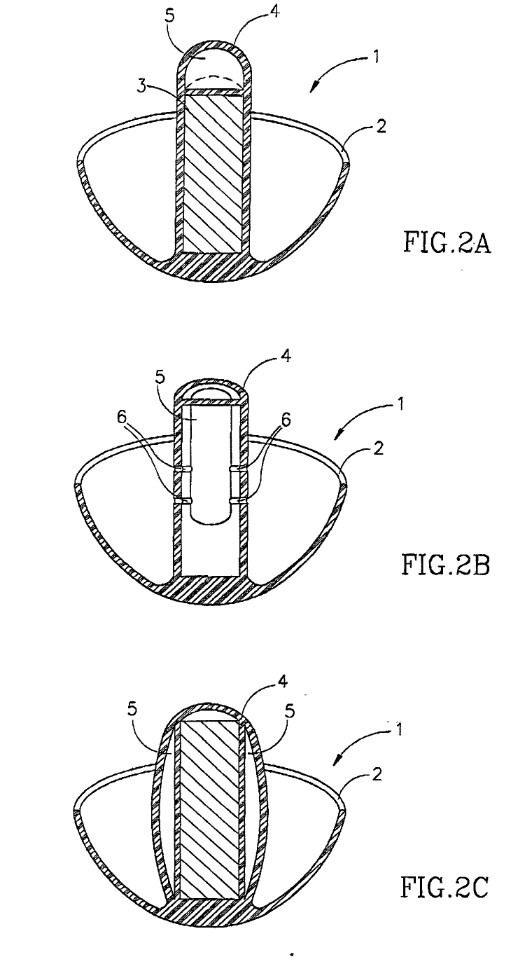

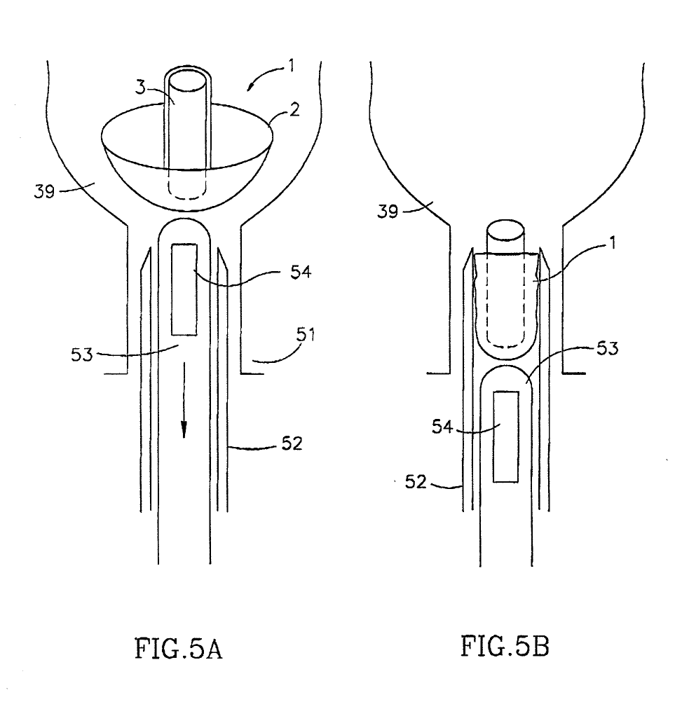

FIG. 2 shows three embodiments of an umbrella in cross section having one or more chambers 5 for storing one or more substances. Such substances could be, for example, drugs, antibiotics or radioactive substances, etc. The chamber 5 may be situated at the tip of the stem, as shown in FIG. 2a. In the embodiment of FIG. 2b, the chamber 5 is located within a hollow magnetable portion 3 and side ports 6 connect the interior of the chamber to the outer surface of magnetable portion 3. In FIG. 5c, chamber 5 is located around the magnetable portion 3. After insertion of the umbrella into the lumen of the urinary bladder, the substances diffuse from the umbrella 1 into the bladder in order to achieve a desired effect.

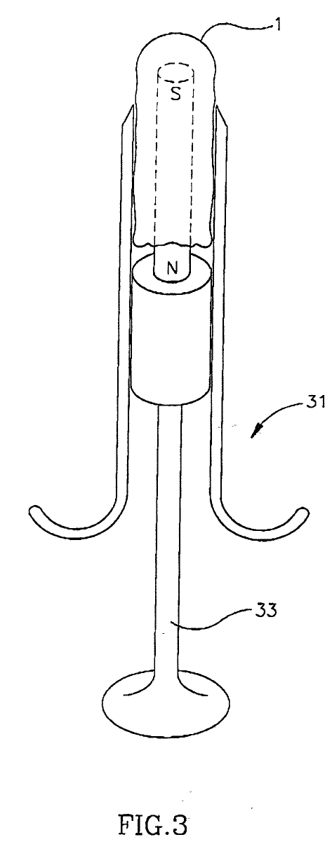

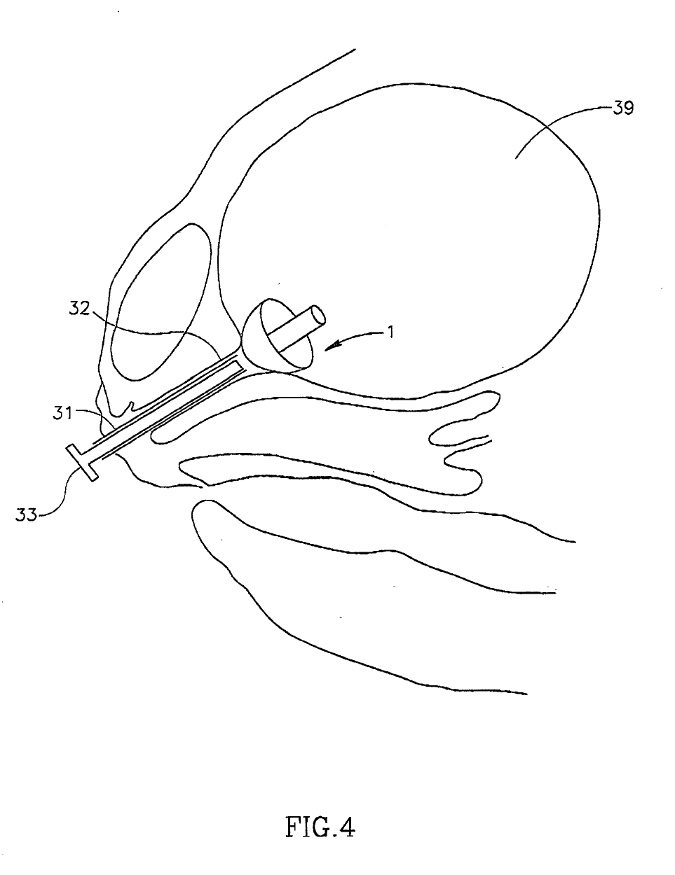

FIG. 3 shows an applicator 31 for inserting an umbrella into the lumen of the urinary bladder of an individual. When umbrella 1 is initially loaded into applicator it is maintained in a deformed state at the end of the applicator. As shown in FIG. 4, the distal end 32 of the applicator-umbrella combination is inserted into the urethra until it reaches the lumen of the bladder. The umbrella 1 is then released from the applicator by pushing the umbrella from applicator 31 with pushing piston 33. The applicator is then removed from the body, leaving the umbrella 1 in the bladder lumen 39. Following its release from the applicator into the bladder, the umbrella regains its initial shape.

FIG. 5a shows a retrieval device generally designated 51 for removing the umbrella from the bladder 39. A catheter 52 has a probe 53 in its lumen which has at its distal end a magnetable portion 54 so as to engage an umbrella 1 at the distal tip by means of the magnetable portion 3 associated with the umbrella. As shown in FIG. 5b, when probe 53 is then retracted, umbrella 1 is deformed and brought into catheter 52. The retrieval device is then withdrawn from the patient together with the umbrella.

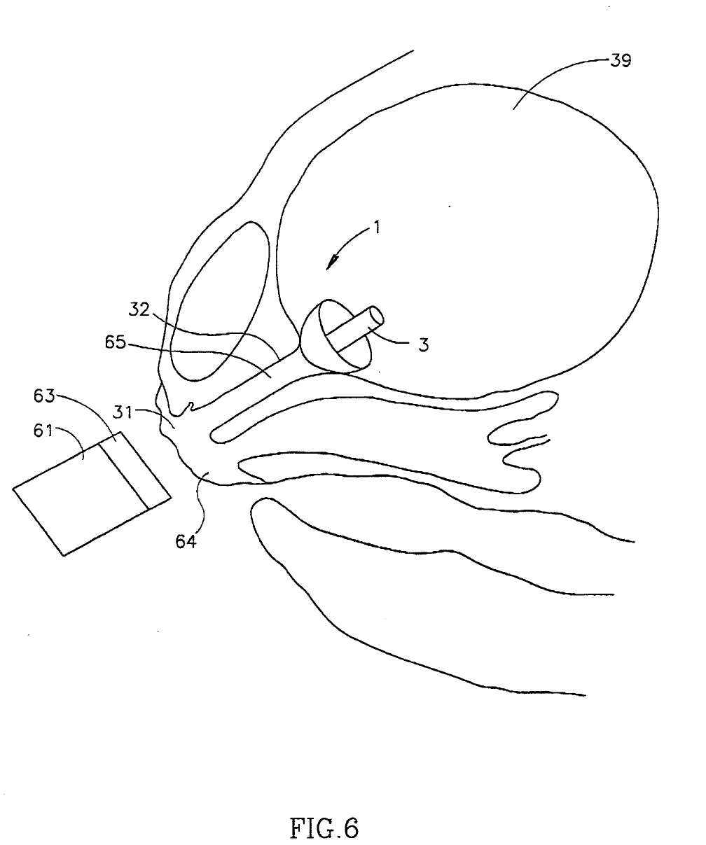

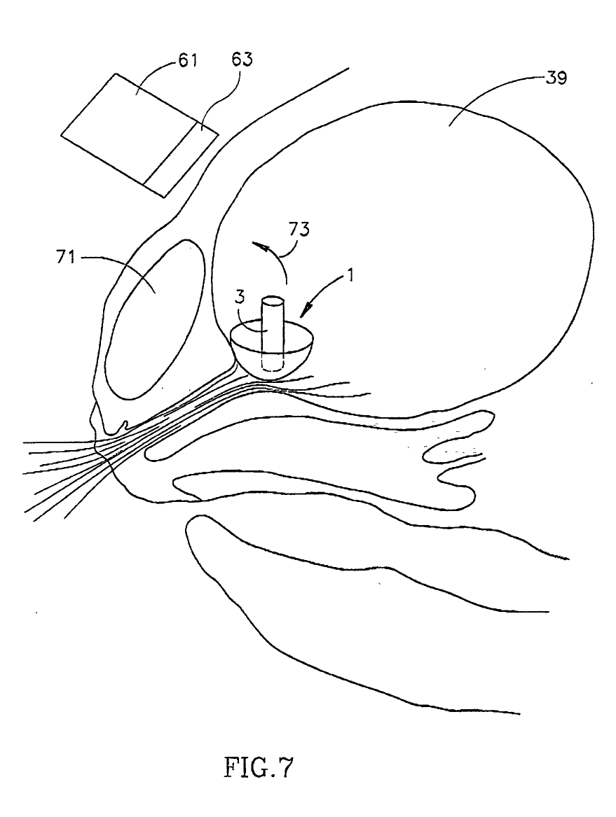

FIGS. 6 and 7 show use of a displacing member 61 to position an umbrella 1 having a magnetable portion 3 at a desired location within the lumen 62 of an individuals urinary bladder. Displacing member 61 is located outside the individual's body and comprises a magnetable portion 63. The displacing member is placed at a location on the surface of the individual's body so as to draw the umbrella from its initial location to the desired location.

FIG. 6 shows use of an umbrella for sealing the urinary bladder outlet in a female subject. Displacing member 61 is placed over the urethral meatus 64 such that, due to the magnetable portion 63 associated with displacing member 61 and the magnetable portion 3 associated with umbrella, the umbrella is drawn into the bladder outlet 65. The umbrella thus becomes lodged in the outlet and seals it. As the amount of urine in the bladder increases, a hydrostatic pressure is exerted on the umbrella further lodging it in the outlet and reinforcing the seal. The invention is used similarly for sealing the urinary bladder owlet in male subjects.

As seen in FIG. 7, in order to open the urinary bladder for voiding, magnetic displacing member 61 is placed over the upper edge of pubic bone 71. Due to the magnetable portion 3 of the umbrella, the umbrella is raised and dislodged from the bladder outlet so as to allow voiding of urine as indicated by arrow 73. After voiding, the umbrella is redrawn into the bladder outlet by the displacing member so as to seal the outlet again as shown in FIG. 6.

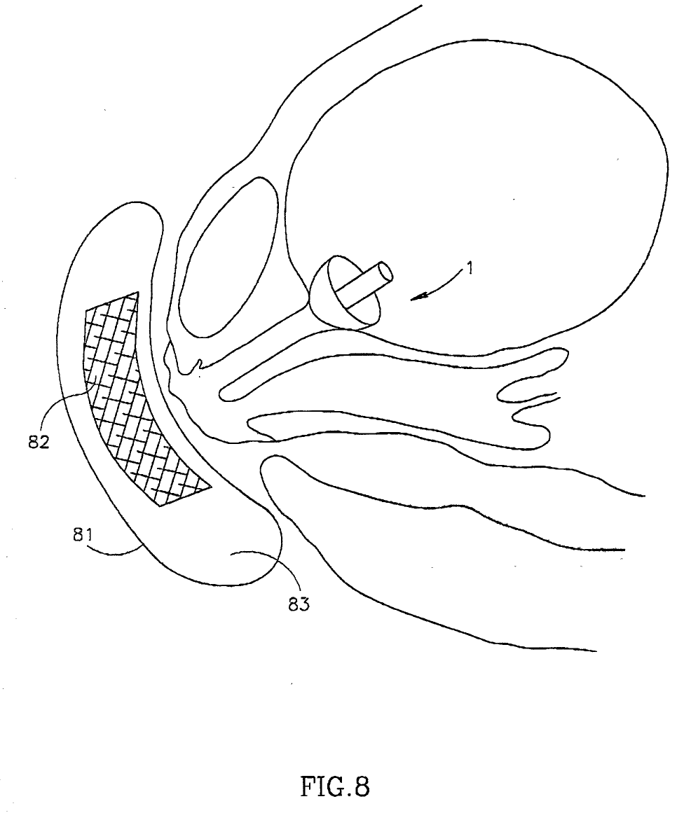

FIG. 8 shows use of an immobilizing member 81 comprising a magnetable portion 82 affixed to the surface of the individual's body so as to maintain umbrella 1 at a desired location in the lumen of the urinary bladder. Magnetable portion 82 of immobilizing member 81 may be enclosed in a coating 83 so as to form, for example, a hygienic pad. The immobilizing member may be affixed to the surface by means of tape, or by pressure applied to it by the individual's underwear.

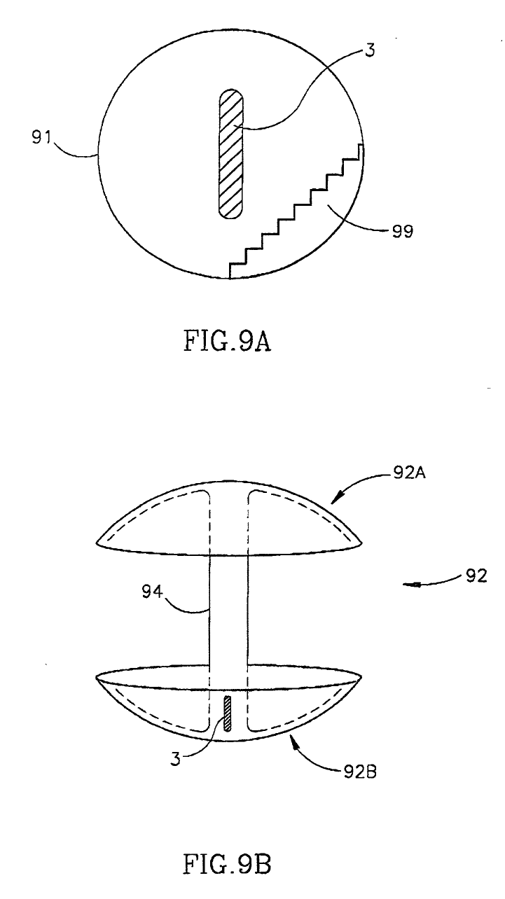

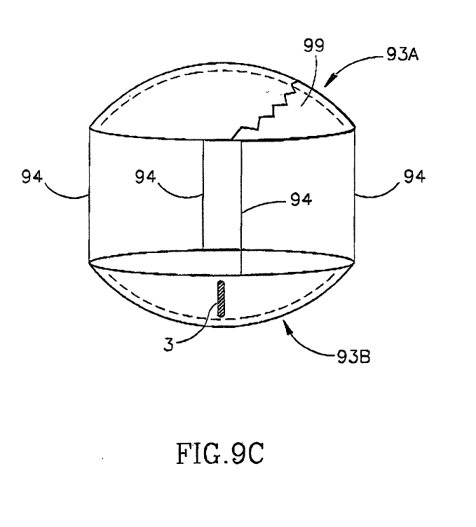

Reference is now made to FIG. 9 which shows three other embodiments of the body for use in the system of the invention. In the embodiment shown in FIG. 9a, the body, generally indicated by 91, has a spherical shape. In the embodiment of FIG. 9b, the body, generally designated as 92, has two hemispherically shaped walls 92a and 92b joined together by a centrally located stem 94. In FIG. 9c, the body, generally indicated by 93, has two hemispherically shaped walls 93a and 93b joined together by a plurality of circumferentially located rods 94. The bodies 91, 92, and 93 may be coated on at least of portion of their outer surface with a hydrophilic coating 99 and may optionally comprise a magnetizable portion 3.

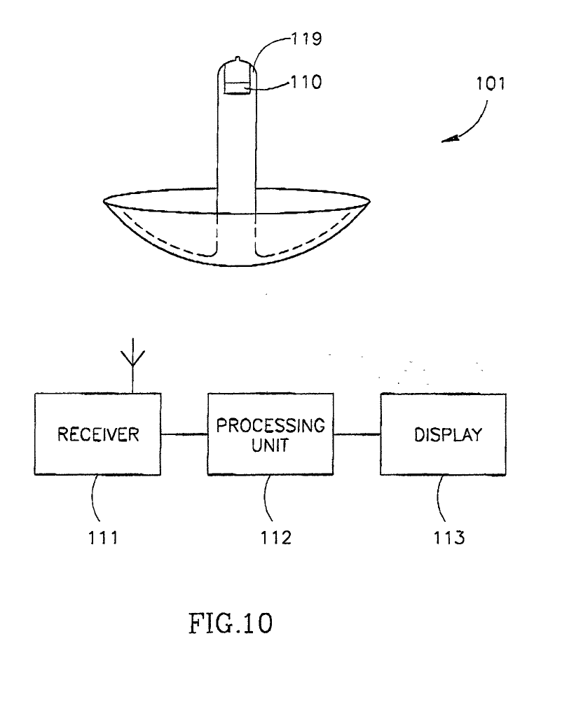

FIG. 10 shows an umbrella 101 constructed so as to comprise an imaging device such as a microvideo camera 119 for imaging the interior of the bladder. The video camera 119 may have associated with it a transmitter 110 for transmitting images to a remote receiver 111. Such microvideo cameras and transmitters are known in the art for example, as disclosed in U.S. Pats. No. 5,604,531, 5,579781 and 5,188,109. The receiver 111 may be connected to a processing unit 112 for processing the images, or a display 113 for displaying images.

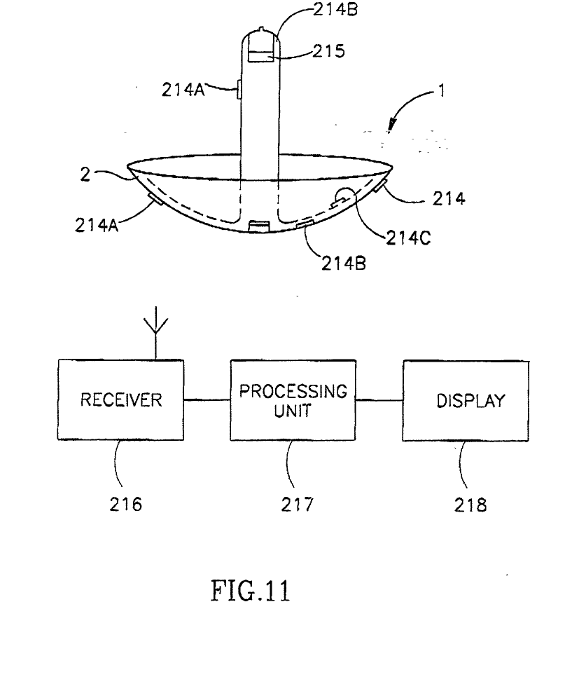

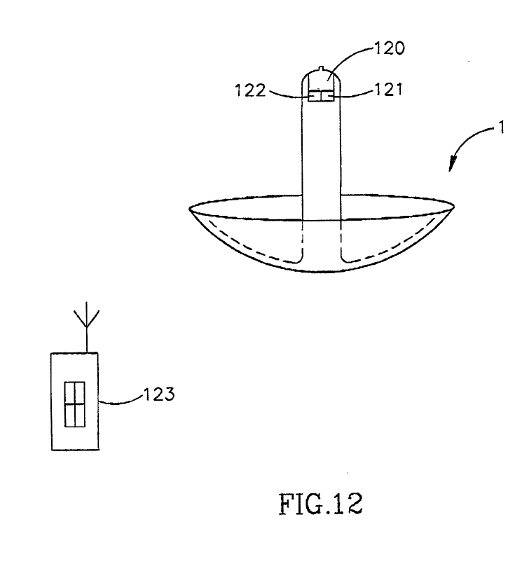

FIG. 11 shows an umbrella 1 constructed so as to comprise one or more devices 214 for measuring one or more parameters associated with the urinary bladder, for example, bladder pressure, urine temperature, urine density, urine conductivity or urine composition. The devices 214 may be affixed to the outer surface of the wall 2 or the stem 4 of the umbrella 214a, embedded within the wall 2 or the stem 4 of the umbrella 214b, or affixed to the inner surface of the wall 214c. The measuring devices 214 may have associated with it a transmitter 215 for transmitting measurements to a remote receiver 216. The receiver may be connected to a processing unit 217 for processing the measurements or to a display 218 for displaying results. Such measuring devices are known in the art, for example as disclosed in U.S. Pats. No. 5,579,781 and 5,188,109.

FIG. 12 shows an umbrella 1 comprising a pump 120 for the controlled release of one or more substances into the bladder. The pump 120 has a reservoir 121 for storing the substances. The pump 120 may have a receiver 122 for receiving signals from a remote control 123. The rate of release of the substance may thus be varied at will using the remote control.

The invention has been described with a certain degree of particularly only for the sake of clarity. However, several variations and modifications in the invention are possible without exceeding the scope and spirit of the invention as defined in the following set of claims.

1. A urological medicinal system for use in medical procedures within a urinary bladder of an individual comprising:
(a) a resiliently flexible, solid body for insertion into the urinary bladder; and
(b) an applicator for inserting the body into the urinary bladder or for removing the body from the bladder, the applicator fitted at an end thereof with a gripping device for releasably gripping the body.

2. The system according to claim 1 wherein the body has a spherical shape.

3. The system according to claim 1 wherein the body comprises a domed-shaped wall and a stem extending from a concave surface of the wall.

4. The system according to claim 1 wherein the body comprises two dome-shaped walls and a centrally located stem having two ends, each end being attached to a concave surface of a different wall.

5. The system according to claim 1 wherein the body comprises two dome-shaped walls and one or more circumferentially located rods, each rod having two ends, each of the two ends of a rod being attached to a concave surface of a different wall.

6. The system according to claim 1, wherein the body is formed with a magnetizable portion.

7. The system according to claim 3, wherein the body is formed with a magnetizable portion consisting of one or more magnetizable particles associated with the stem of the body.

8. The system according to claim 3 wherein the body is formed with a magnetizable portion consisting of one or more magnetizable particles attached to the wall of the body.

9. The system according to claim 3, wherein the body is formed with a magnetizable portion consisting of one or more magnetizable particles embedded in the wall of the body.

10. The system according to claim 4, wherein the body is formed with a magnetizable portion consisting of one or more magnetizable particles associated with the stem of the body.

11. The system according to claim 4 wherein the body is formed with a magnetizable portion consisting of one or more magnetizable particles attached to one or both walls of the body.

12. The system according to claim 4, wherein the body is formed with a magnetizable portion consisting of one or more magnetizable particles embedded in one or both walls of the body.

13. The system according to claim 1 wherein the body is coated on at least a portion of an outer surface with a hydrophilic coating.

14. The system according to claim 1 wherein the body is capable of storing one or more compounds and releasing them into the urinary bladder.

15. The system according to claim 3, wherein the body is capable of storing one or more substances in the wall or the stem and releasing the substances into the urinary bladder.

16. The system according to claim 4, wherein the body is capable of storing one or more compounds in a wall or in the stem and releasing the compounds into the urinary bladder.

17. The system according to claim 9 wherein at least one substance is stored in the wall of the body.

18. The system according to claim 9, wherein at least one substance is selected from the group consisting of antibiotics, anti-microbial agents, drugs, radioactive substances, contrast agents, and substances having a local or systemic effect on cells or tissues of the individual.

19. The system according to claim 1 wherein the body comprises a device which can emit or absorb a signal detectable by an apparatus for imaging the urinary bladder.

20. The system according to claim 19, wherein said device further comprises a transmitter transmitting signals to a receiver.

21. The system according to claim 20 further comprising a receiver receiving signals from the transmitter.

22. The system according to claim 21 further comprising one or more components selected from the list comprising:
(a) a processing unit processing signals received by the receiver;
(b) a display for displaying signals received by the receiver;
(c) a display for displaying an output produced by the processing unit.

23. The system according to claim 1, wherein the body comprises one or more monitoring devices for sampling a morphological or physiological parameter of the urinary bladder and for emitting a signal indicative of a quality or quantity of a sampled parameter.

24. The system according to claim 23, wherein at least one of the devices monitors a parameter of the urinary bladder selected from the group consisting of bladder pressure, urine temperature, urine density, urine conductivity, and urine composition.

25. The system according to claim 23, fixer comprising a transmitter transmitting signals from a monitoring device to a receiver.

26. The system according to claim 25 further comprising a receiver receiving signals from the transmitter.

27. The system according to claim 26, further comprising one or more components selected from the list comprising:
(a) a processing unit processing signals received by the receiver;
(b) a display for displaying signals received by the receiver;
(c) a display for displaying an output produced by the processing unit.

28. The system according to claim 6 further comprising an immobilizing member comprising a magnetizable portion, the immobilizing member being fitted for securing onto the individual's body for immobilizing the body at a desired location in the individual's urinary bladder.

29. The system according to claim 28 wherein the immobilizing member is in the form of a hygienic pad adapted to be placed in the individual's underwear.

30. The system according to claim 1 for use in the treatment of a disorder selected from the group consisting of urinary incontinence, urinary bladder infections, urinary bladder tumors, and bladder dysfunction.

31. The system according to claim 6 further comprising a magnetizable displacing member for displacing the body within the urinary bladder.

32. The system of claim 6 wherein the gripping device comprises a magnetizable portion for securing the balloon thereto during application by means of a magnetic interaction.

33. The system according to claim 20 for use in imaging a urinary bladder.

34. The system according to claim 14 for releasing one or more substances in a urinary bladder.

35. The system according to claim 23 for monitoring a bladder.

36. A method for treating urinary incontinence in an individual comprising the steps of:
(a) compressing a resiliently flexible, solid body, formed with a magnetizable portion;
(b) inserting the body into a urinary bladder of the individual;
(c) decompressing the body in the urinary bladder;
(d) displacing the body into a sealing position for sealing the urinary bladder; and
(e) displacing the body within the urinary bladder into an unsealing position for voiding the urinary bladder.

37. The method according to claim 39 wherein the body is coated on its outer surface with a hydrophilic coating.

38. A method for releasing one or more substances into a urinary bladder comprising the steps of:
(a) loading the one or more substances into a solid, flexibly resilient body;
(b) compressing the body;
(c) inserting the body into the individual's urinary bladder; and
(d) decompressing the body in the urinary bladder.

39. The method of claim 41 further comprising displacing the body within the urinary bladder to a desired location.

40. The method of claim 41 wherein one or more of the one or more substances are selected from the list consisting of antibiotics, anti-microbial agents, drugs, radioactive substances, contrast agents, and substances having a local or systemic effect on cells or tissues of the individual.

41. A method for monitoring the interior of a urinary bladder comprising the steps of:
(a) compressing a solid flexibly resilient body comprising one or more devices for monitoring the urinary bladder;
(b) inserting the body into the individual's urinary bladder; and
(c) decompressing the body in the urinary bladder; and
(d) transmitting signals from at least one of the monitoring devices to a receiver.

42. The method of claim 44 further comprising displacing the body within the urinary bladder to a desired location within the urinary bladder.

43. The method of claim 44, further comprising one or more steps selected from the list comprising:
(a) storing the signals in a computer memory;
(b) displaying the signals on a display;
(c) processing the signals in a computer processing unit;
(d) storing results of the processing in a computer memory; and
(e) displaying results of the processing on a display.

44. A method for imaging the interior of a urinary bladder comprising the steps of:
(a) compressing a flexibly resilient body comprising a device for imaging the urinary bladder;
(b) inserting the body into the urinary bladder;
(c) decompressing the body in the urinary bladder; and
(d) transmitting signals from the imaging device to a receiver.

45. The method of claim 47 further comprising displacing the body within the urinary bladder to a desired location within the urinary bladder.

46. The method of claim 47, further comprising one or more steps selected from the list comprising:
(a) storing the signals in a computer memory;
(b) displaying the signals on a display;
(c) processing the signals in a computer processing unit;
(d) storing results of the processing in a computer memory; and
(e) displaying results of the processing on a display.

47. A method for releasing one or more substances into a urinary bladder comprising steps of
(a) providing a body comprising a pump fed by a reservoir;
(b) loading the reservoir with the one or more substances;
(c) inserting the body into the urinary bladder; and
(d) activating the pump so as to release the one or more substances into the bladder.

48. The method according to claim 44 wherein the pump is activated by a remote control.

49. A resiliently flexible, solid body for insertion into a urinary bladder.

50. The body according to claim 49 comprising one or more magnetizable portions.

51. The body according to claim 49 coated on its outer surface with a hydrophilic coating.

52. The body according to claim 49 capable of storing one or more substances and releasing them into the urinary bladder.

53. The body according to claim 49 comprising a device which can emit or absorb a signal detectable by an apparatus for imaging the urinary bladder.

54. The body according to claim 49 comprising one or more monitoring devices for sampling a morphological or physiological parameter of the urinary bladder and for emitting a signal indicative of a quality or quantity of a sampled parameter.
